(19) United States
(12) Patent Application Publication
(10) Pub. No.: US 2025/0290033 A1
(43) Pub. Date: Sep. 18, 2025

(54) A SCAFFOLDING FOR CULTIVATED MEAT AND A PROCESS FOR MAKING THE SAME

(71) Applicant: MYOWORKS PVT. LTD., Maharashtra (IN)

(72) Inventors: NIHAL SINGH SACHDEVA, Mumbai (IN); NIHAL SINGH SACHDEVA, Mumbai (IN); ANKITA SRIVASTAVA, mumbai (IN)

(21) Appl. No.: 18/271,041

(22) PCT Filed: Mar. 14, 2022
(86) PCT No.: PCT/IN2022/050236

§ 371 Date: Nov. 16, 2023

(30) Foreign Application Priority Data
Mar. 31, 2021 (IN) 202121014874

(51) Int. Cl. C12N 5/00; C12M 1/00; C12M 1/12

(52) U.S. Cl. CPC C12N 5/0068; C12M 23/20; C12M 25/14; C12N 2513/00; C12N 2533/52; C12N 2533/54; C12N 2533/56

(57) ABSTRACT

The present invention relates to an edible scaffolding for cultivated meat made of edible mushroom mycelium. Preferably the scaffolding has a polysaccharide backbone that imparts a 3 dimensional structure and mimics the macroscopic shape of natural meat. The present invention also relates to a process for making the edible scaffolding and the structural backbone. The cultivated meat formed by the process of this invention imitates the texture, shape, appeal, nutrient value and taste as that of natural animal meat.

### FIELD OF INVENTION

The present invention in general, relates to edible scaffolding used in the cultivated meat industry and the process for making the same. Particularly, the present invention relates to a mycelium-composite edible scaffolding and a process for making the same.

### BACKGROUND OF INVENTION

According to World Resources Institute, 2014, As the Global Population increases the demand for food grows unmanageable with every year, “To adequately feed more than 9 billion people by 2050, the world must close a nearly 70 percent gap between the amount of food produced in 2006 and that needed by mid-century.”

Currently, meat, egg and dairy production is highly inefficient from an energy efficiency and demand & supply point of view. In fact, the current system has adversely impacted climate change, environmental damage, and has resulted in antibiotic resistance as the antibiotic-laced meat generates drug resistant bacteria, which in turn is induced into the person consuming such meat.

There is a need for a Sustainable, Efficient and Safe solution to meeting the immense demand of food. This has led to the revolution of “artificial meat”. The industry came up with the first cultivated meat burger in 2013, since then the research and development has been immense.

Particularly, in the following sectors of the artificial meat industry:
Plant based meat
Meat made by fermentation
Cell cultured meat.

While the first two sectors relates of providing a substitute of meat, therefore do not meet the texture and taste demands optimally. Moreover, the third approach relates to producing meat itself but using a method that doesn't involve animal slaughtering. This is achieved by culturing cells obtained from animals and culturing such cells into the desired meat product in laboratories. While the industry has achieved great results, there is a great shortcoming in providing “well-structured” meat. It is found that producing a mass of proliferated cells and serving as minced meat or in the form of a “patty” is achievable. The largest challenge that the industry currently faces lies in producing structured meat parts such as a “meat steak”, “chicken breast”, “fish” or a “drum-stick” etc. having well defined textures as if obtained from the animal itself.

Such a structured form may be obtained by using a scaffold. Moreover, a scaffold is necessary for cells to adhere to and proliferate onto, in addition to its utility in cell culture, cell-based meat scaffolds need to meet additional requirements of edibility, animal free origin and meat-like texture, since one of the central aims of the cell-based meat industry is to redirect the pressure of global demand for protein away from the fragile and often environmentally damaging animal slaughter meat industry (Campuzano, S., & Pelling, A. E., 2019).

The current solutions used in tissue engineering solutions employ animal derived extracellular Matrix (ECM) proteins like Collagen, Fibrin, Fibronectin, or Gelatin as a basis for cell adhesion or synthesized proteins conjugated onto a hydrogel chain to allow for cell adhesion sites that interact with the integrin's in the animal cell. Use of ECM protein to make tissue engineering scaffolds defeats the purpose of cultivated meat to create edible animal tissue without using animals and surface functionalization synthetic peptides or recombinant protein expression systems prove to be prohibitively expensive for the multi-tonne scale of production necessary for this industry.

Also, one option is to culture muscle cells in an appropriate culture medium, the most efficient so far being a medium containing FBS. The medium should provide nutrients, hormones, and growth factors, so that muscle cells will proliferate before being converted into muscle and hence produce a huge amount of meat from a limited number of cells. Hopefully, thanks to technical advances, FBS has been replaced, at least in research laboratories, but may be not yet at the industrial level. Furthermore, as hormone growth promoters are prohibited in conventional farming systems for conventional meat production in the European Union, this is still an issue. However, this technique is able to produce disorganized muscle fibers which are far removed from real muscle, and this is a huge limitation in seeking to reproduce the wide range of meats representing the diversity of animal species and breeds, as well as muscles or cuts. Moreover, the role of blood vessels and blood, nerve tissue, intramuscular fats, and connective tissue affect both taste of meat. Indeed, a number of the “good” veggie meat burgers fail on texture and taste from the point of view of being too uniform. (Sghaier Chriki, 2020)

U.S. Pat. No. 7,270,829B2 provides with a meat product containing in vitro produced animal cells in a three dimensional form and a method for producing the meat product. Moreover, said finished meat product comprises solidified muscle cell tissue as the protein source, wherein the finished meat product is suitable for at least one of human and animal consumption, and wherein the finished meat product is in a form selected from the group consisting of sausage, spread, cooked puree, pureed baby food, biscuit, dried granules, tablet, capsule, powder, pickled meat product, smoked meat product, dried meat product and cooked meat product. But this fails to provide a well-structured meat, maintaining all the nutrient value as well as the texture of the meat that is directly available from the animal. This is the main challenge that the current artificial meat manufacturing industries are facing.

Currently available artificial meat may be cultured over 2D or 3D scaffolds which are unable to offer an orderly formation of cell layers. This is a crucial difference from natural meat, which has highly structured and defined parallel strings of muscle threads. There is therefore a need for an edible scaffold, having meat-like texture, being of animal-free origin and capable of adhesion that can enable cell proliferation, resulting in an orderly formation of structured lab grown meat.

It is therefore an object of this invention to overcome at least one of the shortcomings listed above.

### OBJECT OF THE INVENTION

It is to be noted that the term “meat” in this document may be broadly used to cover fish, shrimp, mollusk, crustacean meat, or any other meat falling under the Animalia kingdom including: vertebrae, Mollusks, Arthropod phyla etc.

It is the primary object of the present invention to make an edible scaffolding for cultivated meat, comprising of one or more cell adhesive component and one or more structural component offering controlled directionality of myotube formation.

Another object of the present invention is to make an edible scaffolding for cultivated meat, comprising of one or more edible adhesive component and a structural component, being an edible polysaccharide backbone.

Yet another object of the present invention is to make an edible scaffolding for cultivated meat free of animal origin while maintaining the nutrient contents and values present in naturally occurring meat.

Yet another object of the present invention is to make an edible scaffolding which mimics natural meat in taste, texture, feel, shape and experience of eating.

Yet another object of the present invention is to make an edible scaffolding, mimicking the dispersed placement of specialized fat tissue as seen in natural meat cuts (commonly referred to as “Marbling” in steak).

Yet another object of the present invention is to make an edible scaffolding offering biomimetic inter-cellular interaction and meat-like texture all desired in the cell-based meat industry.

Yet another object of the present invention is to make an edible scaffolding material that may be 2 dimensional or 3 dimensional.

Yet another object of the present invention relates to a process of making an edible mycelium scaffolding and a process for making an edible polysaccharide backbone for the edible scaffolding.

These and other objects, features and advantages of the present invention will become apparent, to those related to this part of science, in light of the detailed description of the different illustrated embodiments and drawings. The objects enlisted are included in a non-exhaustive, non-limiting manner.

### SUMMARY OF THE INVENTION

Before the present formulation and process, is described, it is to be understood that this application is not limited to the particular disclosure, and details described, as there can be multiple possible embodiments which are not expressly illustrated in the present disclosure. It is also to be understood that the terminology used in the description is for the purpose of describing the particular versions or embodiments only and is not intended to limit the scope of the present application. This summary is provided to introduce concepts related to formulation and processes of making mycorrhizal formulation are further described below in the detailed description. This summary is not intended to identify essential features of the subject matter nor is it intended for use in limiting the scope of the subject matter.

An aspect of the present invention relates to an edible scaffolding material comprising of one or more cell adhesive component, and one or more structural component offering biomimetic linear parallel macrofibrous structure that allows for controlled directionality of myotube formation. Optionally, the cell adhesive component and the structural component may be an edible mycelium.

Another aspect of the present invention relates to an edible scaffolding material comprising of one or more edible adhesive component and one or more structural component being an edible polysaccharide backbone.

Yet another aspect of the present invention relates to an edible scaffolding material which may have a 2 dimensional or 3-dimensional structure such that the 3-dimensional structure is obtained by employing an edible polysaccharide as the structural component.

Yet another aspect of the present invention relates to an edible scaffolding material offering selective negative spaces built into the 3 dimensional scaffolding and wherein the edible scaffolding forms cultivated meat from animal cell cultures.

Yet another aspect of the present invention relates to an edible scaffolding material one or more cell adhesive component, one or more structural component, and a nutrient component comprising.

Yet another aspect of the present invention relates to a process of making edible mycelium scaffolding.

Yet another aspect of the present invention relates to a process for making an edible polysaccharide backbone for the edible scaffolding.

### BRIEF DESCRIPTION OF DRAWINGS

The foregoing detailed description of embodiments is better understood when read in conjunction with the appended drawings. For the purpose of illustrating the disclosure, example constructions of the disclosure are shown in the present document; however, the disclosure is not limited to the specific methods and apparatus disclosed in the document and the drawings.

The detailed description is given with reference to the accompanying figures.

FIG. 1 illustrates a scaffold made using the process of the present disclosure in accordance with an exemplary embodiment.

FIG. 2 illustrates a fish fillet shaped culture dye in accordance with an exemplary embodiment.

FIGS. 3a and 3b illustrates a natural shrimp and shrimp shaped scaffold out of the mycoculture, which is further subjected to cell culture and thereafter administered for MTT assay in accordance with an exemplary embodiment.

FIGS. 4 and 5 illustrate the results from Scanning Electron Microscopy that characterizes the 3D porosity of the scaffolds in accordance with an exemplary embodiment.

FIG. 6 illustrates parallelized strands of adhesive coated, edible polysaccharide backbone for linear bundling and compacting of cultured mycelium of the present disclosure in accordance with an exemplary embodiment.

FIG. 7a illustrates laser scanning confocal microscopy images of animal cells on the scaffold in accordance with an exemplary embodiment.

FIG. 7b illustrates laser scanning confocal microscopy images of animal cells on the shrimp shaped scaffold from various regions showing cell proliferation in accordance with an exemplary embodiment.

FIG. 8a illustrates seeded scaffolds after MTT assay, purple colour indicating presence of cells in accordance with an exemplary embodiment.

FIG. 8b illustrates seeded scaffolds in cell culture media in accordance with an exemplary embodiment.

FIG. 9 illustrates controlled directionality of myotube formation on the polysaccharide backbone in accordance with an exemplary embodiment.

FIG. 10 illustrates making a 3D scaffold in the shape of a shrimp in accordance with an exemplary embodiment.

### DETAILED DESCRIPTION OF THE PRESENT INVENTION

Some embodiments of this disclosure, illustrating all its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise. Although any systems and methods similar or equivalent to those described herein can be used in the practice, the exemplary, systems and methods are now described. The disclosed embodiments are merely exemplary of the disclosure, which may be embodied in various forms. Industry specific terms such as artificial meat/cell-based/in-vitro/lab-grown/cultivated meat shall be read in a non-limiting and an all-encompassing manner in the document.

Various modifications to the embodiment will be readily apparent to those skilled in the art and the generic principles herein may be applied to other embodiments. However, one of ordinary skill in the art will readily recognize that the present disclosure is not intended to be limited to the embodiments illustrated but is to be accorded the widest scope consistent with the principles and features described herein.

Various terms are defined in detail before disclosing the present invention. Here the term “scaffold” means materials that have been engineered to cause desirable cellular interactions to contribute to the formation of new functional tissues for medical purposes. Cells are often ‘seeded’ into these structures capable of supporting three-dimensional tissue formation. The term “porosity” and “negative spacing”/“negative space” have been interchangeably used. Moreover, “mycelium” is the vegetative part of a fungus or fungus-like bacterial colony consisting of a mass of branching, thread-like hyphae. Moreover, MTT stands for (3-(4,5-Dimethylthiazol-2-yl)-2,5-Diphenyltetrazolium Bromide) and MTT assay is a colorimetric assay for assessing the cells metabolic activity. The assay reflects the number of viable cells present in the culture. Also, “Vertebrates” are the class of animals consisting of ‘vertebral column’. These vertebrates include fishes, reptiles, amphibians, birds and mammals. The term “lab grown meat” may be interchangeably used with “artificial meat”, “cultured meat”, “cultivated meat” or such other industrially acceptable terms. “Myotube” means a developmental stage of a muscle fiber composed of a syncytium formed by fusion of myoblasts.

Various embodiments of the present invention relate to an edible mycelium scaffolding and the process of making the same. The edible mycelium scaffolding of the present invention is further used in cell culture of animal cells for the production of lab grown meat. The scaffolding of the present invention is edible, offers optimized animal cell adhesion, while guaranteeing control over the macroscopic shape of the scaffold, and has the ability to be organized to look like a piece of meat while maintaining the structure, shape and texture as that of a natural piece of meat.

An embodiment of the present invention discloses an edible scaffolding for cultivated meat comprising of one or more cell adhesive component, and one or more structural component offering biomimetic linear parallel macrofibrous structure that allows for controlled directionality of myotube formation.

In an embodiment of the present invention the cell adhesive component may be selected from but not limited to a primary adhesive, an adhesive agent, or a combination therefore, wherein the primary adhesive is edible mushroom mycelium and the adhesive agent is Fibronectin, Fibrinogen, Fibroin, Fibrin, Collagen, Matrigel, Vitronectin, Laminin, Poly-L-Lysine or Synthetic peptides/recombinant proteins containing cell adhesion motifs being RGD or LDV.

Another embodiment of the present invention relates to an edible scaffolding material comprising one or more cell adhesive components and an edible structural component, wherein the structural component is an edible polysaccharide backbone offering a 3 dimensional structure to the edible scaffolding.

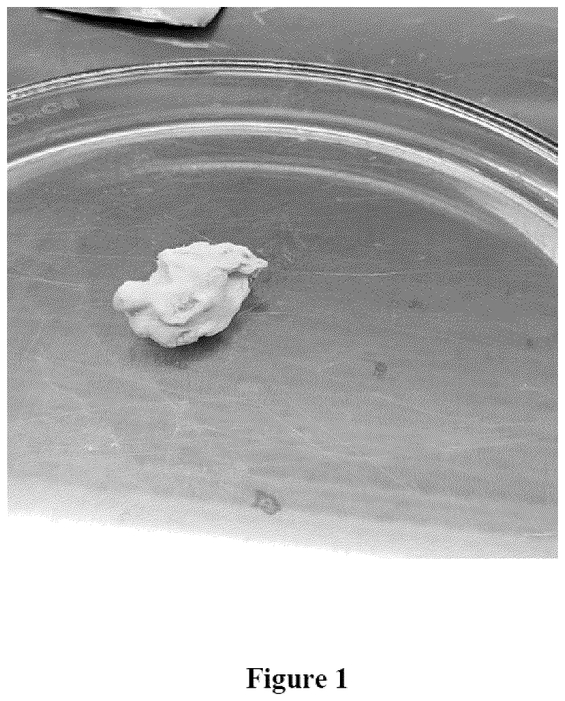

In another embodiment of the present invention the edible polysaccharide backbone enables a 3 dimensional structure of the edible scaffolding which forms a structurally defined framework for the animal cells to be further cultured over. The combination of an edible adhesive component and an edible polysaccharide backbone offers structure to the cultivated meat, wherein the structural component is selected from cellulose, processed plant material, alginate, chitin, chitosan, or a combination thereof. The animal cells which grow over such 3D edible scaffolding have a well-defined structure and resemble meat parts of naturally obtained meat as seen in FIG. 1. The 3D structured edible scaffolding of the present invention further allows selective negative/hollow spaces/porosity to be built into the 3D scaffold, such negative spaces/porosity as seen in FIG. 6, enables mimicking of the dispersed placement of specialized fat tissue in meat cuts, and this is commonly referred to as “Marbling” in steak.

In a further embodiment, the edible scaffolding is post processed treating the mycelium scaffolding with chelating agents such as Sodium Citrate, EDTA, and/or solvents that can selectively dissolve materials in the scaffold to create negative spaces and thereby improve porosity of the mycelium scaffolding.

Yet another embodiment of the present invention relates to the adhesive and structural polysaccharide component, wherein the adhesive component and the structural component are both made of edible mycelium offering a 2 dimensional structure to the edible scaffolding as well as offering selective negative spaces built into the 3 dimensional scaffolding, wherein the 2D and 3D structure to the artificial meat is provided by the adhesive component and the structural component.

A further embodiment of the present invention relates to an edible scaffolding which additionally includes a nutrient component having a solid or liquid form and comprising of cellulosic component, other polysaccharide component(s), glucose, free amino acids, minerals; or a combination thereof.

An even further embodiment of the present invention relates to an edible scaffolding, wherein the edible scaffolding forms cultivated meat from animal cell cultures selected from the vertebrae, Mollusks, Arthropod phyla. Here the vertebrates include animals from fish, reptiles, birds, amphibians and mammals.

An even further embodiment of the present invention relates to a pre-forming process of making the desired shape of mycelium wherein, the mycelium is grown in the desired shape without bruising and damaging the mycelium. Wherein, the mycelium is cultured in a culture die having multiple flow channels and using culture medium containing sugars, peptones, grains, calcium salts; wherein the die is polymeric or metallic with polymeric-coated, non-stick coated, PTFE coated or Teflon coated, and wherein the mycelium is cultured at a temperature range of 20-30° C., maintaining 4-20% CO2 at 100% humidity level.

After culturing the mycelium in a culture die, the strands of the mycelium are organized and a linear bundle of mycelium is formed after compacting the formed strands of mycelium.

An even further embodiment of the present invention relates to a process of making an edible mycelium scaffolding comprising of following steps:
a) Prepare the nutrient media to receive mycelium starter;
b) Culturing the mycelium starter on a culture die with the nutrient medium for 5-20 days to form the mycelium scaffolding;
c) Removing the mycelium scaffolding from the nutrient media and cleaning the scaffolding;
d) Arresting the mycelium growth by industrially accepted drying techniques (air-drying or freeze-drying techniques);
e) Subjecting the mycelium scaffolding to UV radiation for sterilization;
f) Treating the mycelium scaffolding with alcohol to make it hydrophilic;
g) Drying the mycelium scaffolding to remove all traces of alcohol and preparing for cell culture.

An additional embodiment of the present invention provides a detailed process for making the scaffold, which comprises of the following steps:
a) Pre-treat mycoculture with 70% ethanol for 3 h to prepare it for cell-culture (add enough ethanol to completely cover the Scaffold).
b) Remove ethanol from the scaffold.
c) Air-dry scaffolds till ethanol evaporates, then wash three times with PBS.
d) Leave scaffold in the third PBS wash and expose the scaffolds to UV radiation for 30 minutes.
e) Aspirate the PBS and add 1000 μl complete medium per well.
f) Incubate the scaffolds with medium at 37° C. overnight. Proceed with seeding after this step.

An additional embodiment of the present invention relates to a process of culturing the mycelium starter on a culture die, wherein the culture die contains a polysaccharide backbone formed by steps as follows:
a. Extracting or creating polysaccharide fibers;
b. Arranging said fibers; and
c. Reconstituting the polysaccharide fibers using mycelium invasion to form mycelium scaffolding having a polysaccharide backbone.

An additional embodiment of the present invention further relates to a process of seeding the mycelium scaffold with animal cells, wherein the process includes:
a) Seeding animal cells directly onto the center of the mycelium scaffold for optimum cell attachment;
b) Incubating the seeded scaffold in a humidified incubator at 20-40° C. with 4-20% carbon dioxide for at least 3 hours to facilitate cell attachment and form edible scaffolding for cultivated meat;

For optimal results, cells should be seeded directly onto the center of the scaffold in small volumes of appropriate media to allow cell attachment.

In yet another embodiment of the present invention, after the edible mycelium scaffold is subjected to cell culturing, and once the cells start invading the mycelium, the cells are subjected to MTT ((3-(4,5-dimethylthiazol-2-yl)-2,5-diphenyltetrazolium bromide) tetrazolium reduction) assay. MTT assay is a colorimetric assay for assessing cell metabolic activity. Thus, it indicates the presence or absence of cells.

Yet another embodiment of the present invention relates to a method for the characterization of the 3D porosity of the scaffold. Scanning Electron Microscopy is used for the characterization of the porosity of the scaffold. It was found that nutrient values maintain a good porosity. Thus, maintaining and modifying the nutrient values help to maintain a good porosity. A good porosity of the edible scaffold maintains the texture and look of the artificial meat.

Yet another embodiment of the present invention relates to culturing artificial meat and giving the meat the shape selected from but not limited to shrimp, fish, chicken breasts, steak and fillets.

### EXPERIMENTAL DATA

The present disclosure is not intended to be limited to the experiments illustrated below but is to be accorded the widest scope consistent with the principles and features described herein.

### Experiment 1

A piece of cultivated meat, made using the present invention was tested for various “natural meat-like” characteristics:
1. Texture
2. Shape
3. Parallel macro fibrous structure

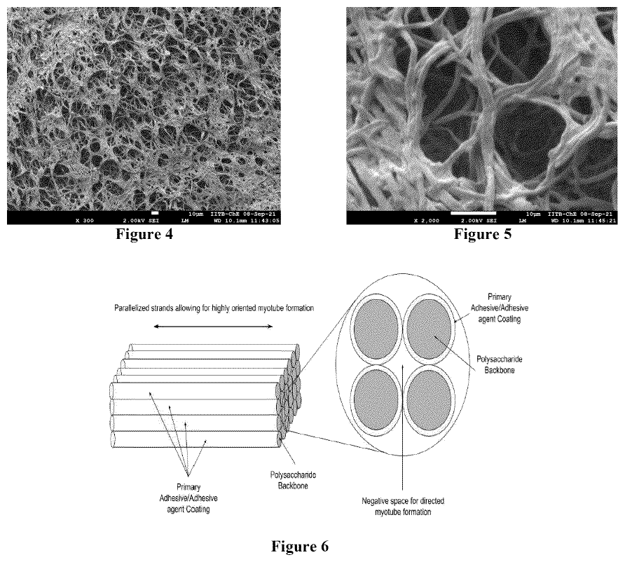

From Scanning Electron Microscopy (SEM) experiment 300× and 2000× magnification images were taken as seen in FIGS. 4 and 5 the images showed distinct “negative spaces” or “porosity” as found in natural meat and therefore highly desirable in cultivated meat. It is this porosity that defines the shape and texture of the meat, and importantly offers biomimetic linear parallel macrofibrous structure that allows for controlled directionality of myotube formation. The experimental data showed negative spaces.

### Experiment 2

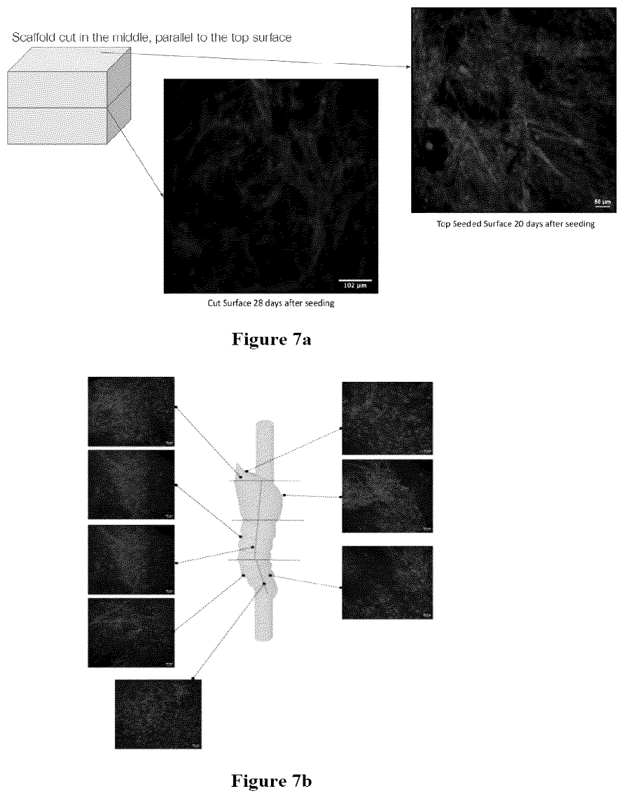

Cell migration of animal cells cultured over the edible scaffolding of the present invention was studied to understand if the cells are able to travel through the scaffold, proving the porosity of the scaffold. (As shown in FIGS. 7a and 7b)

### Experiment 3

A shrimp was made employing the edible scaffolding of the present invention, following the steps detailed in Examples 1 & 2. The final product as seen in FIG. 3b was compared with a natural shrimp and the two were found to be strikingly similar in texture as well as appeal (see FIG. 3a).

### Experiment 4

Cell adhesion of animal cells over the edible scaffolding of the present invention was studied to understand if the cells are able to attach on the scaffold. MTT assay performed on the cultured cells on the edible scaffolding proved adhesion of the cells to the scaffold. The result showed 71.51-77.97% adhesion efficiency of cells to the scaffold.

The process as detailed in example 5 was followed for adhesion of chick embryo fibroblast on the scaffold.

The results for the cell adhesion experiment are shown in the below table:

| | | Number of Cells | |
|---|---|---|---|
| | | adhered to | |
| Number of Total | Number of Live | scaffold | Adhesion |
| Cells in flushing | Cells in flushing | (Calculated) | Efficiency of |
| media (# Cells) | media (# Cells) | (# Cells) | Scaffold (%) |
| 90000 | 80000 | 270000 | 75.00% |
| 79320 | 72000 | 280680 | 77.97% |
| 102560 | 98900 | 257440 | 71.51% |
| 99000 | 91000 | 261000 | 72.50% |

### EXAMPLES

### Example 1

Part I—Making an Edible Mycelium Scaffolding for Chicken (Gallus gallus Domesticus) Breast:
a. A mycoculture nutrition media containing glucose, free amino acids, mineral salts and a cellulosic component was selected;
b. Pre-processed Polysaccharide fibers were arranged and placed on the chicken breast culture die;
c. Chicken breast shaped culture die made of metal and coated with Teflon, was selected and placed on top of mycoculture nutrition media;
d. Cultured 4 mm of mycelium starter cells in the center of the polysaccharide fibers present on the culture die and incubated it for 10 days to form edible chicken breast mycelium scaffold;
e. Arrested the mycelium growth by Air-drying techniques;
f. Sterilized the mycelium scaffold using gamma radiation in the 25-30 kGy;
g. Treated the edible mycelium scaffolding with 70% ethanol for 3 h to prepare it for cell culture (added enough ethanol to completely cover the Scaffold);
h. Removed ethanol from the scaffold;
i. Evaporated the ethanol, and washed it three times with PBS;
j. Left the scaffold in the third PBS wash and exposed the scaffold to UV radiation for 30 minutes.

### Part II—Cultivating a Chicken Breast Piece on an Edible Mycelium Scaffolding:

a. Aspirated the PBS and added 1000 μl of complete medium per well;
b. Incubated the scaffolds with the complete medium at 4° C. overnight;
c. Seeded 1 million cells of chicken breast cells in the center of the chicken breast mycelium scaffold, incubated at 37° C. with 5% CO2 for at least 3 hours to facilitate cell attachment;
d. Cultured over 28 days with regular media replenishments to create cultivated chicken breast product.

### Example 2

### Part I—Making an Edible Mycelium Scaffolding for Chicken Meat Patty:

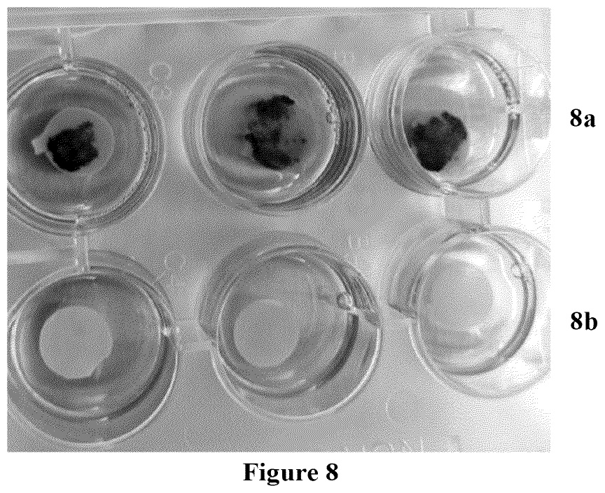

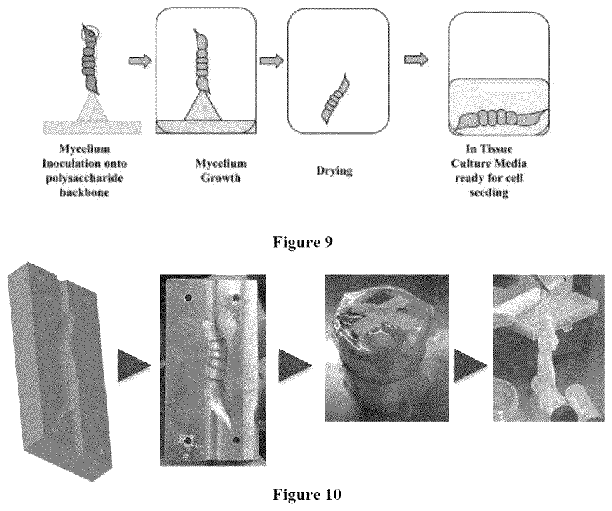

a. A nutrient media containing glucose, free amino acids, mineral salts and a polysaccharide component;
b. A petri dish (see FIG. 8) made of plastic was selected and placed in 3 ml liquid nutrient media;
c. Cultured 1 mm of mycelium starter cells on the culture die and incubated it for 8 days to form 2D edible mycelium scaffold; (see FIG. 8)
d. Separating the mycelium from the growth medium and drying the mycelium and arrested the mycelium growth by lyophilisation technique;
e. Sterilized the mycelium scaffold using gamma radiation in the 25-30 kGy;
f. Treated the edible mycelium scaffolding with 70% ethanol for 3 h to prepare it for cell culture (add enough ethanol to completely cover the Scaffold);
g. Air dry the mycelium until ethanol evaporated, and washed it three times with PBS;
h. Left the scaffold in the third PBS wash and exposed the scaffold to UV radiation for 30 minutes.

### Part II—Cultivating Chicken Meat Patty on an Edible Mycelium Scaffolding:

a. Aspirated the PBS and added 1000 μl of complete medium per well;
b. Incubated the scaffolds with the complete medium at 4° C. overnight;
c. Seeded 20 μl of shrimp cells in the center of the chicken patty mycelium scaffold, incubated at 32° C. with 17% CO2 for at least 4 hours 100% humidity level to facilitate cell attachment;
d. Performed MTT assay on the scaffold, to administer the viability of the cells.

### Example 3

### Making an Edible Mycelium Scaffolding for Fish:

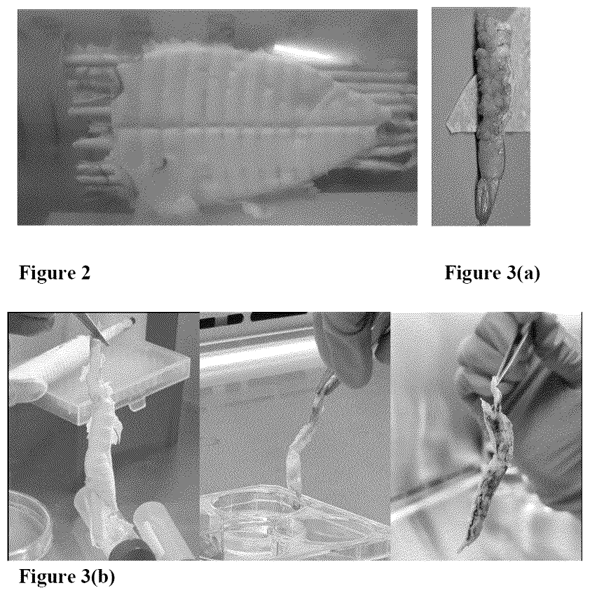

a. A nutrient media containing glucose, free amino acids, minerals, cellulosic component was prepared to receive the mycelium starter;
b. Pre-processed Polysaccharide fibers extracted from alginate were arranged and placed on the fish culture die;
c. Fish shaped culture die (see FIG. 2) made of metal coated with PTFE was selected and placed on top of mycoculture nutrition media;
d. Cultured 5 mm of mycelium starter cells in the center of the polysaccharide fibers present on the culture die and incubated it for 12 days to form edible fish mycelium scaffold;
e. Arrested the mycelium growth by Air-drying techniques;
f. Sterilized the mycelium scaffold using gamma radiation in the 25-30 kGy;
g. Treated the edible mycelium scaffolding with 70% ethanol for 3 h to prepare it for cell culture (add enough ethanol to completely cover the Scaffold);
h. Evaporated the ethanol from the scaffold;
i. Arrested the mycelium growth by freeze-drying technique until ethanol evaporated, and washed it three times with PBS;
j. Left the scaffold in the third PBS wash and exposed the scaffold to UV radiation for 30 minutes.

### Example 4

### Making an Edible Mycelium Scaffolding for Shrimp:

a. A mycoculture nutrition media containing glucose, free amino acids, mineral salts and a cellulosic component was selected;
b. Pre-processed Polysaccharide fibers extracted from alginate were arranged and placed on the shrimp culture die;
c. Shrimp shaped culture die made of metal and coated with PTFE, was selected and placed on top of mycoculture nutrition media;
d. Cultured 4 mm of mycelium starter cells in the center of the polysaccharide fibers present on the culture die and incubated it for 7 days to form edible shrimp mycelium scaffold (see FIG. 10);
e. Arrested the mycelium growth by Air-drying techniques;
f. Sterilized the mycelium scaffold using gamma radiation in the 25-30 kGy;
g. Treated the edible mycelium scaffolding with 70% ethanol for 3 h to prepare it for cell culture (added enough ethanol to completely cover the Scaffold);
h. Removed ethanol from the scaffold;
i. Evaporated the ethanol, and washed it three times with PBS;
j. Left the scaffold in the third PBS wash and exposed the scaffold to UV radiation for 30 minutes.

### Example 5

### Adhering Chicken Embryo Fibroblast on the Edible Mycelium Scaffold

a. Seeded each scaffold in 12 well plate with 100,000 cells in a cell suspension of 20 μl.
b. Incubated the seeded scaffolds for 4 hours.
c. Flushed each well with 1 ml media and rinsed each scaffold with media using pipette 10 times such that non-adhered cells will dissociate off the scaffold and are suspended in the flushing media.
d. Moved the scaffold to a different well, centrifuged the flushing media at 3000 rpm for 5 minutes followed by a cell count using Trypan Blue and the countess FL3.
e. Performed MTT on the rinsed scaffold 1 day later to show that adhered cells remained viable.

The results for the cell adhesion experiment are shown table given in Experiment 4.

### Advantages of the Present Invention

A few advantages of the edible scaffolding of the present invention are disclosed here below. It is stated here that the below mentioned list is non-limiting and is provided to give a broad understanding of the novel properties of the present invention without limiting its scope or understanding.
1. The present invention enables mimicking of the dispersed placement of specialized fat tissue in meat cuts; this is commonly referred to as “Marbling” in steak.
2. The edible scaffolding of the present invention is capable of biomimetic inter-cellular interaction.
3. The scaffolding of the present invention is free of animal origin.
4. The adhesive component of the present invention may be made of recombinant protein or synthetic peptides.
5. The edible scaffolding meets additional requirements of edibility, animal free origin and meat-like texture all desired in the cell-based meat industry.
6. Moreover, the present invention also maintains the nutrient contents and values present in the naturally occurring meat.

1. A scaffolding for cultivated meat comprising of:
one or more cell adhesive component, and
one or more structural component offering biomimetic linear parallel macrofibrous structure that allows for controlled directionality of myotube formation.

2. The scaffolding as claimed in claim 1, wherein the cell adhesive component is a primary adhesive, an adhesive agent, or a combination thereof.

3. The scaffolding as claimed in claims 1 and 2, wherein the primary adhesive is edible mushroom mycelium and the adhesive agent is Fibronectin, Fibrinogen, Fibroin, Fibrin, Collagen, Matrigel, Vitronectin, Laminin, Poly-L-Lysine or Synthetic peptides/recombinant proteins containing cell adhesion motifs being RGD or LDV.

4. The scaffolding as claimed in claim 1, wherein the structural component is preferably an edible polysaccharide backbone offering a 3 dimensional structure to the edible scaffolding.

5. The scaffolding as claimed in claims 1 and 4, wherein the structural component is edible and made of cellulose, processed plant material, alginate, chitin, chitosan, or a combination thereof.

6. The scaffolding as claimed in claim 1 wherein the adhesive component and the structural component are both made of edible mycelium offering a 2 dimensional structure to the edible scaffolding.

7. The structural component as claimed in claims 1,-6, offering selective negative spaces built into the 3 dimensional scaffolding.

8. The scaffolding as claimed in claim 1, additionally includes a nutrient component having a solid or liquid form and comprising of cellulosic component, other polysaccharide component(s), glucose, free amino acids, minerals; or a combination thereof.

9. The scaffolding as claimed in claim 1, wherein the scaffolding is edible and forms cultivated meat from animal cell cultures selected from the vertebrae, Mollusks, Arthropod phyla.

10. A process of making a scaffolding for cultivated meat comprising the following steps:
a. Prepare the nutrient media to receive mycelium starter;
b. Culturing the Mycelium starter on a culture die with the nutrient medium for 5-20 days to form the mycelium scaffolding;
c. Removing the mycelium scaffolding from the nutrient media and cleaning the scaffolding;
d. Arresting the mycelium growth by industrially acceptable drying technique;
e. Subjecting the mycelium scaffolding to radiation for sterilization;
f. Treating the mycelium scaffolding with alcohol to make it hydrophilic;
g. Drying the mycelium scaffolding to remove all traces of alcohol and preparing for cell culture.

11. The process of making a scaffolding for cultivated meat as claimed in claim 10 wherein the process additionally includes Culturing the Mycelium starter on a culture die, wherein the culture die contains a polysaccharide backbone formed by steps as follows:
a. Extracting or creating polysaccharide fibers;
b. Arranging said fibers; and
c. Reconstituting the polysaccharide fibers using mycelium invasion to form a mycelium scaffolding having a polysaccharide backbone.

12. The process of making a scaffolding for cultivated meat as claimed in claims 10 and 11 wherein the process further includes:
a. Seeding animal cells directly onto the center of the mycelium scaffold for optimum cell attachment;
b. Incubating the seeded scaffold in a humidified incubator at 20-40° C. with 4-20% CO₂ for at least 3 hours to form edible scaffolding for cultivated meat.

13. The process of making a scaffolding as claimed in claims 10-12 wherein the process further includes treating the mycelium scaffolding with chelating agents such as Sodium Citrate, EDTA, and/or solvents that can selectively dissolve materials in the scaffold to create negative spaces and thereby improve porosity of the mycelium scaffolding.

14. The process of making a scaffolding as claimed in claims 10-13 wherein the process further includes Culturing Mycelium as follows:
a. Culturing Mycelium in a culture die having multiple flow channels and using culture medium containing sugars, peptones, grains, calcium salts;
wherein the die is polymeric or metallic with polymeric-coated, non-stick coated, PTFE coated or Teflon coated; and
b. Ensuring a temperature range of 20-30° C., maintaining 4-20% CO₂ at 100% humidity level.
